(19) United States
(12) Patent Application Publication
(10) Pub. No.: US 2012/0082695 A1
(43) Pub. Date: Apr. 5, 2012

(54) COSMETIC ACTIVE PREPARATION

(75) Inventor: Marcus Asam, Unterfoehring (DE)

(73) Assignees: AXEL RUTH, Unterfoehring (DE); ARJAM ASAM, Unterfoehring (DE)

(21) Appl. No.: 12/954,891

(22) Filed: Nov. 28, 2010

(30) Foreign Application Priority Data
Nov. 27, 2009 (DE) 10 2009 055 970.1

(51) Int. Cl. A61K 8/97; A61Q 19/00

(52) U.S. Cl. 424/195.17

(57) ABSTRACT

A cosmetic formulation comprises extracts and/or their derivatives, i.e., sea aster, brown algae, coral algae and Irish moss. In addition, metal-bound peptides are also contained in the cosmetic formulation. These components of the cosmetic formulation reinforce each other mutually so that the entire formulation exhibits favorable moisturizing effects that soothe the skin and increase its elasticity. Furthermore, a skin treated with it is very largely protected from damaging external environmental influences.

The invention relates to a cosmetic formulation used in particular for skin cream.

The invention has the problem of creating a cosmetic formulation that is distinguished by a moisturizing action that soothes the skin and increases its elasticity, whereby the skin is additionally protected from damaging external environmental influences.

The invention solves this problem with the following features.

The cosmetic formulation comprises extracts and/or their derivatives from sea plants and sea algae.

The sea aster (Tripleurospermum maritima) grows preferably on sparse sandy hills on the Atlantic Breton coast. This sea plant is rich in vitamins B5, B6, B3 and B9 as well as in mineral substances. Furthermore, it contains a high concentration of essential amino acids. These substances offer great protection to the skin against free radicals and thus have an antioxidant action. Moreover, they have the property of reducing inflammatory processes in the skin, which reduces the formation of spots on the skin. The extract is preferably obtained from the sea aster in such a manner that the above-cited substances are contained in the highest-possible concentration in the extract.

The brown algae Alaria esculenta comes preferably from Brittany and is rich in unsaturated fatty acids, in particular omega 3, omega 6 and omega 9 fatty acids. Furthermore, it contains plant sterols and lipoamides distinguished by their antioxidant action. Active substances contained in brown algae have a stimulating action in the skin for the synthesis of glycosaminoglycanes, hyaluronic acid and proteoglycane. Therefore, this counteracts the degradation of elastin and collagen and in this manner improves the elasticity of the skin. Furthermore, they increase the activity of sirtuins, that prevent aging of the skin. The extract is preferably obtained in such a manner from the brown algae in such a manner that the above-cited substances are contained in the highest-possible concentration in the extract.

Coral algae (Corralina officinalis) is especially rich in important trace elements and minerals which are of decisive importance for the activation of enzymes in the skin so that the enzyme formation furthered by the above-cited extracts also results in an increased enzymatic activity. The extract is preferably obtained from the coral algae in such a manner that the above-cited substances are contained in the highest-possible concentration in the extract.

Irish moss (Chondnrus chrispus (carrageen)) contains in particular a natural biopolymer that acts as a film former and comes to rest in a protective manner on the uppermost skin layers. This improves in particular the sensory perception of the skin. The skin feels soft to the touch and appears more uniform. Thus, this active-substance component additionally protects the skin from damaging environmental influences and improves its ability to store moisture. The extract from Irish moss is preferably obtained in such a manner that the above-cited substances are contained in the highest-possible concentration in the extract.

Finally, the cosmetic formulation contains peptides still bound to metallic particles. These peptides preferably have an amino acid sequence similar to TGF-β. Whereas the previously cited active substance components all have a natural biological origin, this component is artificially synthesized. This additional active substance component has a significant effect on the extracellular matrix, that is, the intermediate cellular spaces. Furthermore, this active substance significantly increases the natural collagen synthesis. This reduces the formation of deep wrinkles and significantly improves the elasticity of the skin. As a result of the bonding of the peptides to metallic particles the peptides can be brought into the skin in an especially high concentration in order to degrade even deeply located wrinkles. This results in an especially high anti-ageing effect. The metallic particles preferably have an average size of at the most 2 μm, whereby the use of nanoparticles is preferred. This facilitates the penetration of the particles into the skin. The bonding preferably takes place in a coordinated manner in the form of clusters or covalently, preferably via sulfide bridges or disulfide bridges.

It turned out that the individual above-cited components have a significant interaction with each other. Thus, for example, the radical-trapping effect produced by the sea aster extract and by the Irish moss extract is potentiated by the peptides bound to the metallic particles. As a result, the cost-intensive biological extracts can be used in a considerably lesser concentration than without the peptide reinforcers. Furthermore, the effect of the bound peptides is reinforced by the Irish moss extract and the coral algae extract.

It turned out in a manner worthy of notice that the above-described cosmetic formulation has a significant muscle-relaxing effect which far exceeds that which can be expected from sea water extract. All remaining active substances were basically supposed to have no muscle-relaxing effect. It is suspected here that metallic particles have, after giving off peptides bound to them, a reinforcing effect on the sea aster extract. The muscle-relaxing effect is very positive for the cosmetic formulation since it also reduces wrinkles.

In particular platinum or a platinum alloy have proven themselves for the metallic particles. Platinum or platinum alloys have the property of preventing the degradation of peptides in the skin by enzymes. Furthermore, platinum or platinum alloy particles also protect enzymes from the biological extracts from an uncontrolled degradation.

The following values have proven themselves for the concentration of the individual active substance components:

1% to 50%, preferably between 5% and 30% for the sea aster extract.
10% to 90% preferably between 20% and 70% for the brown algae extract.
2% to 50%, preferably between 5% to 30% for the coral algae extract.
2% to 30%, preferably between 5% to 15% for the Irish moss extract, and
2% to 60%, preferably between 4% to 30% for peptides bound to metallic particles.

All percentage data is to be understood as percentage by weight.

### EXAMPLES

### 1. Facial Cream

A facial cream consists of 54.6% water, 6% moisture-retaining factor, 6% fatty phase thickeners, 23.8% oils, 0.5% thickeners and 3.65% formulation.

The formulation itself is composed of the following active substances:

27% brown algae extract, 27% coral algae extract, 14% Irish moss extract, 28% sea aster extract and 4% peptides bound to platinum particles.

This face cream was subjected to a dermatological investigation which used 30 test persons between 50 and 70 years old with healthy skin in the testing area. The test persons had no chronic skin inflammations. Care was taken that the test persons did not take any medicaments that could adversely affect the skin reactions. Sunbathing and the visiting of solariums were excluded during the carrying out of the study.

The test persons used the above-described face cream daily in a given area that had been examined prior to the start of the test study and after one month by optical 3-D measuring of the wrinkle depth.

The following table shows in the first column the number of test persons, in the second column the measured wrinkle depth before using the facial cream and in the third column the measured wrinkle depth after one month of using the facial cream. Finally, the fourth column shows the computer-determined percentage of wrinkle depth according to the formula: wrinkle depth after/wrinkle depth before minus 1.

| | μm | μm | % |
|---|---|---|---|
| 1 | 158.9 | 73.0 | −54.1% |
| 2 | 166.2 | 83.2 | −49.9% |
| 3 | 191.1 | 93.3 | −51.2% |
| 4 | 152.3 | 65.3 | −57.1% |
| 5 | 164.4 | 85.5 | −48.0% |
| 6 | 170.7 | 80.9 | −52.6% |
| 7 | 192.3 | 76.0 | −60.5% |
| 8 | 167.4 | 69.7 | −58.4% |
| 9 | 159.0 | 73.1 | −54.0% |
| 10 | 175.5 | 86.8 | −50.5% |
| 11 | 168.8 | 80.2 | −52.5% |
| 12 | 171.4 | 89.1 | −48.0% |
| 13 | 205.7 | 100.2 | −51.3% |
| 14 | 170.6 | 86.6 | −49.2% |
| 15 | 156.2 | 58.2 | −62.7% |
| 16 | 171.2 | 69.6 | −59.3% |
| 17 | 164.7 | 71.9 | −56.3% |
| 18 | 185.0 | 85.5 | −53.8% |
| 19 | 181.3 | 83.8 | −53.8% |
| 20 | 192.9 | 113.7 | −41.1% |
| 21 | 162.9 | 68.7 | −57.8% |
| 22 | 173.0 | 80.8 | −53.3% |
| 23 | 154.7 | 59.8 | −61.3% |
| 24 | 167.9 | 82.4 | −50.9% |
| 25 | 170.6 | 74.6 | −56.3% |
| 26 | 156.4 | 56.7 | −63.7% |
| 27 | 191.3 | 100.8 | −47.3% |
| 28 | 179.4 | 99.4 | −44.6% |
| 29 | 154.6 | 63.4 | −59.0% |
| 30 | 153.3 | 55.0 | −64.1% |
| | | | −54.1% |

The determined percentage of wrinkle depth was determined for all test persons in order to be able to make a statement that could be statistically evaluated. Accordingly, an average reduction of wrinkle depth of 54.1% of the original wrinkle depth results.

### 2. Test Formulation A

This test formulation A corresponds to the facial cream according to point 1, whereby, however, the peptides bound to platinum particles were omitted without replacement. This reference cream A accordingly contains only the algae extracts as active substance.

The use of test formulation A took place at the same time with the facial cream according to point 1 by the same test persons but in another area. This other area was also measured before and one month after the use in the same manner as in the case of the face cream according to point 1. The results of this measuring are shown in the following table.

| | μm | μm | % |
|---|---|---|---|
| 1 | 159.1 | 118.6 | −25.5% |
| 2 | 178.6 | 133.8 | −25.1% |
| 3 | 189.4 | 156.3 | −17.5% |
| 4 | 158.0 | 116.9 | −26.0% |
| 5 | 177.4 | 128.6 | −27.5% |
| 6 | 171.2 | 130.7 | −23.7% |
| 7 | 193.1 | 132.4 | −31.4% |
| 8 | 157.9 | 125.6 | −20.5% |
| 9 | 146.3 | 118.3 | −19.1% |
| 10 | 182.6 | 128.2 | −29.8% |
| 11 | 169.7 | 137.3 | −19.1% |
| 12 | 168.7 | 131.4 | −22.1% |
| 13 | 205.8 | 156.4 | −24.0% |
| 14 | 173.1 | 126.3 | −27.0% |
| 15 | 156.6 | 116.0 | −25.9% |
| 16 | 174.6 | 140.6 | −19.5% |
| 17 | 170.3 | 127.3 | −25.2% |
| 18 | 188.6 | 148.2 | −21.4% |
| 19 | 189.0 | 151.4 | −19.9% |
| 20 | 181.0 | 142.6 | −21.2% |
| 21 | 161.9 | 118.0 | −27.1% |
| 22 | 167.7 | 135.4 | −19.3% |
| 23 | 156.9 | 120.9 | −22.9% |
| 24 | 160.6 | 126.5 | −21.2% |
| 25 | 175.0 | 137.1 | −21.7% |
| 26 | 158.3 | 108.7 | −31.3% |
| 27 | 200.0 | 161.9 | −19.1% |
| 28 | 190.2 | 137.0 | −28.0% |
| 29 | 159.1 | 111.8 | −29.7% |
| 30 | 147.5 | 107.6 | −27.1% |
| | | | −24.0% |

An average reduction of wrinkle depth of 24/0% results from this test series that is therefore distinctly below the reduction of wrinkle depth of the facial cream according to point 1.

### 3. Test Formulation B

The same composition as in the case of the facial cream according to point 1 was used as a further test formulation B, whereby exclusively the peptides bound to platinum particles were used in the same concentration as active substance. Even the test formulation B was tested by the same test persons as for the facial cream according to point 1 under the same test conditions, whereby again another skin area was used. The results of this study are collated in the following table.

| | μm | μm | % |
|---|---|---|---|
| 1 | 156.9 | 126.0 | −19.7% |
| 2 | 164.7 | 151.7 | −7.9% |
| 3 | 196.9 | 163.6 | −16.9% |
| 4 | 165.9 | 127.3 | −23.3% |
| 5 | 162.8 | 134.1 | −17.6% |
| 6 | 159.0 | 135.8 | −14.6% |
| 7 | 203.7 | 162.3 | −20.3% |
| 8 | 164.1 | 139.7 | −14.9% |
| 9 | 161.6 | 148.9 | −7.9% |
| 10 | 176.1 | 155.5 | −11.7% |
| 11 | 161.3 | 127.5 | −21.0% |
| 12 | 183.2 | 139.6 | −23.8% |
| 13 | 223.0 | 185.6 | −16.8% |
| 14 | 174.9 | 149.5 | −14.5% |
| 15 | 152.4 | 120.0 | −21.3% |
| 16 | 169.4 | 150.0 | −11.5% |
| 17 | 156.6 | 133.7 | −14.6% |
| 18 | 194.0 | 160.6 | −17.2% |
| 19 | 174.0 | 134.4 | −22.8% |
| 20 | 186.4 | 160.0 | −14.2% |
| 21 | 157.8 | 122.6 | −22.3% |
| 22 | 187.1 | 158.2 | −15.4% |
| 23 | 153.2 | 120.0 | −21.7% |
| 24 | 169.6 | 146.7 | −13.5% |
| 25 | 171.3 | 152.3 | −11.1% |
| 26 | 152.9 | 115.7 | −24.3% |
| 27 | 184.6 | 157.4 | −14.7% |
| 28 | 183.3 | 140.4 | −23.4% |
| 29 | 166.5 | 133.4 | −19.9% |
| 30 | 155.5 | 134.8 | −13.3% |
| | | | −17.1% |

An average reduction of wrinkle depth of 17.1% results from this study. Even this value is considerably below that of the facial cream according to point 1. It is noteworthy that the sum of the reductions of wrinkle depth of the two test formulations A and B with 41/1% is distinctly below the action of the reduction of wrinkle depth of the facial cream according to point 1 (54.1%). The determined reduction of wrinkle depth is accordingly approximately 32% above the expected value. This proves that the algae extracts with the peptides bound to platinum particles enter a synergistic effect by means of which the effect of the total formulation can be increased beyond a pure summing effect. This synergetic effect is not yet understood at the present time and was also not expected.

Accordingly, the facial cream according to point 1 has a surprisingly strong wrinkle-reducing effect and in addition an extremely high antioxidant effect was demonstrated.

### 4. Facial Serum

A facial serum consists of 80.7% water, 7.85% moisture-retaining factors, 3.5% thickeners, 2.6% oils, 0.3% emulsifiers and 1.7% formulation.

The formulation itself consists for its part of:

59% brown algae extract, 6% coral algae extract, 12% Irish moss extract, 6% sea aster extract and 17% peptides bound to platinum particles.

This facial serum is distinguished by a high anti-wrinkle effect and a high antioxidant effect.

### 5. Eye Cream

An eye cream consists of 64.3% water, 16% moisture-retaining factors, 0.75% emulsifiers, 1.95% fatty phase thickeners, 5% oils and 1.7% formulation. The formulation itself consists of: 59% brown algae extract, 6% coral algae extract, 6% Irish moss extract, 6% sea aster extract and 23% peptides bound to platinum particles.

This eye cream has an unexpectedly high effect that improves the elasticity of the skin as well as has a very high anti-wrinkle effect.

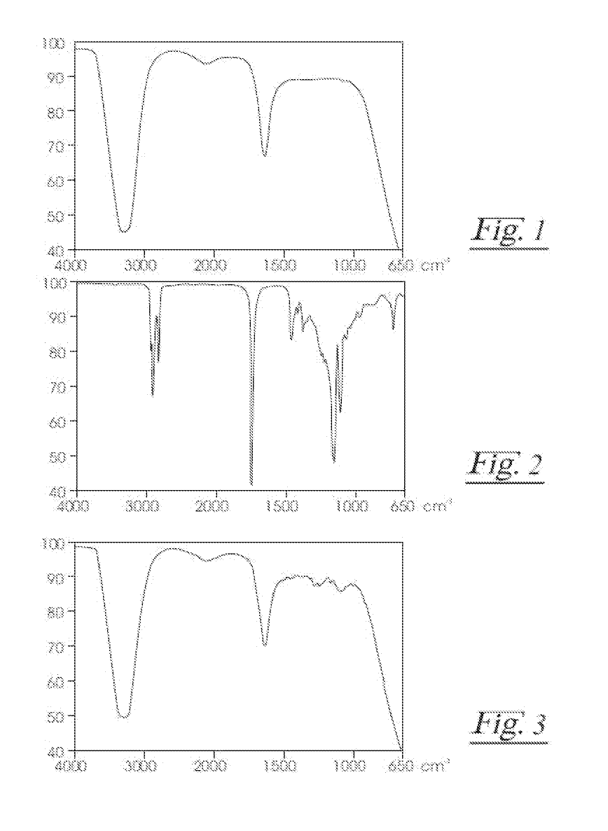

FIG. 1 shows an infrared spectrum of the sea water extract used in the examples.

FIG. 2 shows an infrared spectrum of the brown algae extract used in the examples.

FIG. 3 shows an infrared spectrum of the coral algae extract used in the examples.

1. A cosmetic formulation comprising the following components:
a) at least one substance selected from an extract and from a derivative of an extract from a sea aster,
b) at least one substance selected from an extract and from a derivative of an extract from a brown algae,
c) at least one substance selected from an extract and from a derivative of an extract from a coral algae,
d) at least one substance selected from an extract and from a derivative of an extract from an Irish moss, and
e) peptides bound to metal particles.

2. The cosmetic formulation according to claim 1, whereby it contains vitamins B3, B5, B6 and/or B9.

3. The cosmetic formulation according to claim 1, whereby it contains essential amino acids.

4. The cosmetic formulation according to claim 1, whereby it contains at least one of the unsaturated fatty acids omega 3, omega 6 and omega 9.

5. The cosmetic formulation according to claim 1, whereby it contains at least one of the following substances, plant sterols lipoamides and trace elements.

6. The cosmetic formulation according to claim 1, whereby the brown algae extract is obtained from Alaria esculenta.

7. The cosmetic formulation according to claim 1, whereby the coral algae extract is obtained from Corralina officinalis.

8. The cosmetic formulation according to claim 1, whereby the metal particles contain platinum.

9. The cosmetic formulation according to claim 1, whereby the total amount consisting of the sea aster extract and its derivative is contained in the formulation in a concentration between 1% and 50%.

10. The cosmetic formulation according to claim 9, whereby the total amount consisting of the sea aster extract and its derivative is contained in the formulation in a concentration between 3% and 30%.

11. The cosmetic formulation according to claim 1, whereby the total amount consisting of the brown algae extract and its derivative is contained in the formulation in a concentration between 10% and 90%.

12. The cosmetic formulation according to claim 11, whereby the total amount consisting of the brown algae extract and its derivative is contained in the formulation in a concentration between 20% and 70%.

13. The cosmetic formulation according to claim 1, whereby the total amount consisting of the coral algae extract and its derivative is contained in the formulation in a concentration between 2% and 50%.

14. The cosmetic formulation according to claim 13, whereby the total amount consisting of the coral algae extract and its derivative is contained in the formulation in a concentration between 3% and 30%.

15. The cosmetic formulation according to claim 1, whereby the total amount consisting of the Irish moss extract and its derivative is contained in the formulation in a concentration between 2% and 30%.

16. The cosmetic formulation according to claim 15, whereby the total amount consisting of the Irish moss extract and its derivative is contained in the formulation in a concentration between 5% and 15%.

17. The cosmetic formulation according to claim 1, whereby the peptides bound to metal particles are contained in a concentration between 2% and 60%.

18. The cosmetic formulation according to claim 17, whereby the peptides bound to metal particles are contained in a concentration between 4% and 30%.

19. The cosmetic formulation according to claim 1, whereby at least one of the extracts is obtained at least partially from an organism stemming from Brittany.
